(19) United States
(12) Patent Application Publication
(10) Pub. No.: US 2025/0334116 A1
(43) Pub. Date: Oct. 30, 2025

(54) PISTON PUMP STABILIZER

(71) Applicant: Insulet Corporation, Acton, MA (US)

(72) Inventor: Kyle Breingan, Westford, MA (US)

(21) Appl. No.: 19/188,150

(22) Filed: Apr. 24, 2025

Related U.S. Application Data
(60) Provisional application No. 63/638,168, filed on Apr. 24, 2024.

(51) Int. Cl. F04B 53/14; A61M 5/315

(52) U.S. Cl. CPC F04B 53/143; A61M 5/31513; F04B 53/144

(57) ABSTRACT

A delivery system of an automated medicament delivery device for automated administration of medicament to a user-body is disclosed. The delivery system includes a reservoir and a delivery mechanism. The reservoir includes a sidewall defining an internal volume therein. The delivery mechanism includes a piston and a rod. The piston is received in the internal volume and includes a body, a seal, and at least one protrusion. The seal is received around a perimeter of the body and forms a seal between the body and the sidewall. The at least one protrusion extends from the body beyond the perimeter and towards the sidewall. The rod connects to the body. The delivery mechanism is configured to cause a friction bias between the seal and the sidewall around the perimeter of the body during movement of the piston relative to the reservoir.

### CROSS-REFERENCE TO RELATED APPLICATIONS

This application claims the benefit under 35 U.S.C. § 119 (e) of U.S. Provisional Patent Application Ser. No. 63/638,168, filed Apr. 24, 2024, the disclosure of which is hereby incorporated herein in its entirety by this reference.

### TECHNICAL FIELD

This disclosure relates generally to automated medicament delivery devices. More particularly, the present disclosure relates to stabilizers for pumps for medicament delivery systems.

### BACKGROUND

Automated medicament delivery devices (A M D, e.g., Automated Insulin Delivery (AID) device, without limitation) are often used to administer medicaments from a reservoir of the AMD to the body of a patient via a cannula inserted into the body to treat medical conditions (e.g., Type 1 Diabetes, without limitation).

In AMD piston pumps, inconsistencies or unsteady movement of the drive mechanism may cause the piston to wobble during movement thereof in the reservoir, which may result in the piston “walking” down the reservoir. This unsteady movement may be caused by symmetric friction between the piston and reservoir. The symmetric friction on all sides of the piston may put the system in an unstable environment, so any wobble introduced from the drive will allow the piston to walk. The unsteady movement and walking behavior of the piston while moving in the reservoir may cause inconsistent drug delivery volumes as the piston shifts back and forth during pulsing. This may cause pulse-to-pulse inaccuracy in dose size or inaccuracies every few pulses as the plunger tilts at different angles over time.

### BRIEF SUMMARY

In one illustrative embodiment, the present disclosure provides a delivery system of an automated medicament delivery device for automated administration of medicament to a user-body. The delivery system includes a reservoir and a delivery system. The reservoir includes a sidewall defining an internal volume therein. The delivery mechanism includes a piston and a rod. The piston is received in the internal volume. The piston includes a body, a seal, and at least one protrusion. The seal is received around a perimeter of the body and extends outward beyond the perimeter and forming a seal between the body and the sidewall. Each of the at least one protrusion extends from the body beyond the perimeter and towards the sidewall. The rod connects to the body. The delivery mechanism is configured to cause a friction bias between the seal and the sidewall around the perimeter of the body during movement of the piston relative to the reservoir.

In another illustrative embodiment, the present disclosure provides a delivery system of an automated medicament delivery device for automated administration of medicament to a user-body. The delivery system includes a reservoir and a delivery mechanism. The reservoir includes a sidewall defining an internal volume therein. The delivery mechanism includes a piston and a rod. The piston is received in the internal volume. The piston includes a body, a seal, one or more first protrusions and one or more second protrusions. The body includes a connection end and a pressure end, opposite the connection end. The seal received around a perimeter of the body and extending outward beyond the perimeter and forming a seal between the body and the sidewall. The one or more first protrusions extend from a first side of the body adjacent to the connection end extend toward the sidewall. The one or more second protrusions extend from a second side of the body, opposite the first side, adjacent to the pressure end and extend toward the sidewall. The rod connected to the body at a connection point on the connection end, the connection point being offset from a center of the body to the first side of the body.

In a further illustrative embodiment, the present disclosure provides a delivery system of an automated medicament delivery device for automated administration of medicament to a user-body. The delivery system includes a reservoir and a delivery mechanism. The reservoir includes sidewalls defining an internal volume therein. The delivery mechanism includes a piston received in the internal volume. The piston includes a body, a seal, one or more first protrusions, and one or more second protrusions. The body includes a connection end and a pressure end, opposite the connection end. The body includes an asymmetrical shape with a first side and a second side. A first perimeter of the first side of the body including a length that is longer than a length of a second perimeter of the second side of the body. The seal is received around a combined perimeter of the body and extending outward beyond the combined perimeter and forming a seal between the body and the sidewall. The one or more first protrusions extend from the first side of the body adjacent to the connection end and extend toward the sidewall. The one or more second protrusions extend from a second side of the body, opposite the first side, adjacent to the pressure end and extend toward the sidewall.

### BRIEF DESCRIPTION OF THE DRAWINGS

For a detailed understanding of the present disclosure, reference should be made to the following detailed description, taken in conjunction with the accompanying drawings, in which like elements have generally been designated with like numerals, and wherein:

FIG. 1 is a schematic diagram illustrating an automated medicament delivery device;

FIG. 2 is a block diagram of a medicament delivery system 200 for controlled administering of medicament, in accordance with one or more examples;

FIG. 3 is a cut-away perspective view of a portion of a delivery system in accordance with one or more embodiments;

FIG. 4 is a cut-away side view of the portion of the delivery system of FIG. 3;

FIG. 5 is a top view of the portion of the delivery system of FIG. 3;

FIG. 6 is a cut-away perspective view of a portion of a delivery system in accordance with one or more embodiments;

FIG. 7 is a cut-away side view of the portion of the delivery system of FIG. 6; and

FIG. 8 a top view of the portion of the delivery system of FIG. 6.

### DETAILED DESCRIPTION

In various embodiments, a stabilized delivery system (for delivering medicament) of an AMD is disclosed. In particular, the delivery system is configured to apply a torque to a piston while the piston is moved within and relative to a reservoir of the delivery system. The torque may be applied by adding a friction bias to the piston, which may prevent walking/wobbling of the piston as the piston moves within the reservoir. As will be described in detail below, protrusions are positioned on the piston to stabilize and maintain alignment of the piston with the reservoir and to counteract the moment applied to the piston by the torque resulting from the friction bias while medicament is being administered.

The illustrations presented herein are not actual views of any system, device, or structure, or any component thereof, but are merely idealized representations, which are employed to describe embodiments of the present invention.

As used herein, the singular forms following “a,” “an,” and “the” are intended to include the plural forms as well, unless the context clearly indicates otherwise.

As used herein, the term “may” with respect to a material, structure, feature, or method act indicates that such is contemplated for use in implementation of an embodiment of the disclosure, and such term is used in preference to the more restrictive term “is” so as to avoid any implication that other compatible materials, structures, features, and methods usable in combination therewith should or must be excluded.

As used herein, any relational term, such as “first,” “second,” “top,” “bottom,” “upper,” “lower,” “above,” “beneath,” “side,” “upward,” “downward,” without limitation, is used for clarity and convenience in understanding the disclosure and accompanying drawings, and does not connote or depend on any specific preference or order, except where the context clearly indicates otherwise. For example, these terms may refer to an orientation of elements of any system, device, or structure, when utilized in a conventional manner. Furthermore, these terms may refer to an orientation of elements of any system, device, or structure, as illustrated in the drawings.

As used herein, the term “substantially” in reference to a given parameter, property, or condition means and includes to a degree that one skilled in the art would understand that the given parameter, property, or condition is met with a small degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

As used herein, the term “about” used in reference to a given parameter is inclusive of the stated value and has the meaning dictated by the context (e.g., it includes the degree of error associated with measurement of the given parameter, as well as variations resulting from manufacturing tolerances, without limitation).

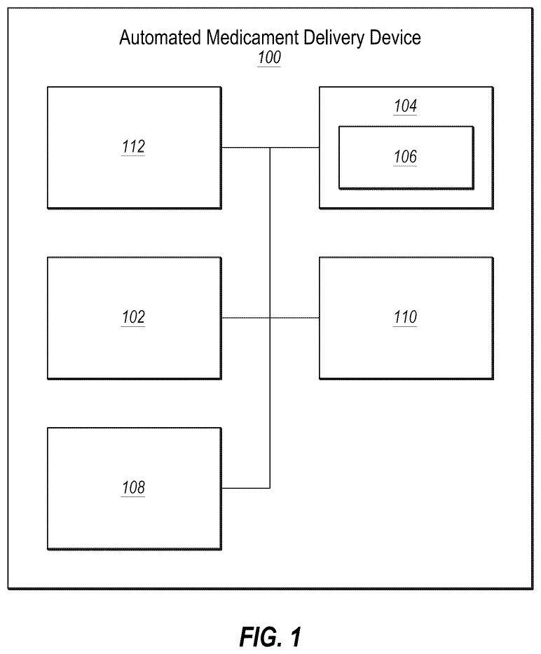

FIG. 1 is a schematic diagram illustrating an automated medicament delivery device 100 for automated administration of medicament to a user-body, in accordance with one or more embodiments.

In one or more embodiments, the automated medicament delivery device 100 may be capable of one or more operative modes of administration of medicament. Non-limiting examples of the one or more operative modes include: fully automated administration of medicament, partially automated administration of medicament, or manual administration of medicament. In one or more embodiments, the automated medicament delivery device 100 may be capable of alternating between multiple (e.g., two or more, without limitation) operative modes. As a non-limiting example, the automated medicament delivery device 100 may alternate between one or more of: fully automated operation, partially automated operation, and manual operation.

The automated medicament delivery device 100 may administer medicament at least partially based on one or more values representative of amounts of one or more analytes present within a user-body (such values respectively an “analyte value”). The one or more analytes may include constituents of the user-body and foreign substances, such as medicaments, markers, metabolites, and combinations or subcombinations of one or more of the foregoing, without limitation. The automated medicament delivery device 100 may also administer an amount of medicament at least partially based on user inputs (e.g., a user defined bolus amount or details related to a meal consumed or about to be consumed, such as number of carbohydrates, amount of fat, and amount of protein, without limitation).

Non-limiting examples of medicaments administrable by the automated medicament delivery device 100 include: insulin, glucagon-like peptide-1 receptor agonist (GLP-1), glucose-dependent insulinotropic polypeptide (GIP), pramlintide, or other hormones, insulin substitutes, and combinations of medicaments, such as two or more of insulin, GLP-1, and GIP, or other like hormones. While specific examples discussed herein may involve insulin or GLP-1, or GIP, this disclosure is not limited to those examples, and other medicaments do not exceed the scope. As a non-limiting example, glucagon, morphine, analgesics, fertility medicaments, blood pressure medicaments, chemotherapy drugs, arthritis drugs, weight loss drugs, without limitation, are non-limiting examples of medicaments that are specifically contemplated.

The automated medicament delivery device 100 is configured to administer medicament to a user-body, such as subcutaneously into the user-body, without limitation, in accordance with one or more embodiments. In one or more embodiments, the automated medicament delivery device 100 may offer one or more operative modes for administration of medicament to a user-body. When operating in some of the operative modes, automated medicament delivery device 100 may administer medicament at least partially responsive to analyte values, including without limitation, analyte values received from an analyte sensor. The analyte sensor is configured to obtain data related to one or more analytes within the user-body (“analyte data”). The analyte sensor may be an analytical bio-sensing device, such as a continuous glucose monitor (CGM) or an integrated continuous glucose monitor (ICGM) (e.g., examples of commercially available analytical bio-sensing devices include the FREESTYLE LIBRE® 3 manufactured by Abbott or the DEX COM® G6 manufactured by Dexcom, without limitation).

When operating in some further operative modes, automated medicament delivery device 100 may administer medicament at least partially responsive to user input. When operating some yet further operative modes, automated medicament delivery device 100 may administer medicament at least partially responsive to analyte values and user input. Non-limiting examples of the one or more operative modes offered by automated medicament delivery device 100 include: fully automated administration of medicament, partially automated administration of medicament, or manual administration of medicament. When operating in an operative mode that includes manual administration of medicament, automated medicament delivery device 100 may administer medicament solely in response to a user input (e.g., delivers medicament in response to a user confirmation of delivery of medicament or in response to a user instruction to delivery medicament, without limitation). When operating in an operative mode that includes fully automated administration of medicament, automated medicament delivery device 100 may administer medicament solely in response to analyte values (e.g., delivers medicament in response to one or more analyte values, without limitation). When operating in an operative mode that includes partially automated administration of medicament, automated medicament delivery device 100 may administer medicament in response to analyte values and user input (e.g., delivers medicament in response to a user input and an analyte value, or alternately delivers medicament in response to a user input or in response to analyte values, without limitation). Medicament administration may include administration of a basal amount of medicament regularly delivered a control interval (e.g., at a determined basal rate, without limitation) to keep analyte levels stable and within a determined or predetermined range. Medicament administration may also include administration of bolus amounts of medicament administered as an immediate bolus, an extended bolus, or a combination bolus (combination of an immediate bolus and an extended bolus). The bolus amount of medicament may be a correction bolus responsive to a change in analyte levels or a user defined bolus (e.g., responsive to user inputs provided, such as a user defined bolus amount or details related to a meal consumed or about to be consumed, such as number of carbohydrates, amount of fat, and amount of protein, without limitation).

The automated medicament delivery device 100 includes a delivery system 112, one or more processors 102, memory 104, communication equipment 108, and a power source 110. In one or more embodiments, the automated medicament delivery device 100, or portions thereof, may be a wearable device and may be secured to a user-body (e.g., secured via one or more adhesive layers attaching the automated medicament delivery device 100 to the skin of the user-body or a material that is secured to the user-body, without limitation).

In various embodiments, the delivery system 112 is configured to cause an amount of medicament to move (e.g., flow, without limitation) toward and/or into a user-body.

In various embodiments, delivery system 112 may deliver amounts of medicament at least partially responsive to requests. In various embodiments, instructions 106 of memory 104 may include instructions for determining and generating requests for delivery system 112. In various embodiments, instructions 106 may include instructions for determining one or more amounts of medicament, determining a timing for delivery of one or more amounts of medicament, and for generating one or more requests for delivery system 112 related to the same. When such instructions of instructions 106 are executed by one or more processors 102, the one or more processors 102 determine the amounts of medicament and timing of delivery, generate requests for the delivery system 112 at least partially based on the determined amounts and timing, and provide the requests to delivery system 112.

The communication equipment 108 is configured to facilitate communication (e.g., wireless communication, without limitation) of the automated medicament delivery device 100 with other devices, including without limitation, communication between the automated medicament delivery device 100 and the analyte sensor and/or a controller (e.g., a dedicated electronic device, a smart phone, a tablet computer, a wearable device, without limitation). The communication may be wired or wireless communication and may utilize any suitable communication protocol such as wireless networking protocol (e.g., Wi-Fi®, without limitation), a short-range wireless protocol (e.g., BLUETOOTH®, without limitation), a near-field communication standard, a cellular standard, or any other wireless optical or radio-frequency protocol. In various embodiments, the communication equipment 108 includes an Internet of Things (IoT) Subscriber Identity Module (SIM) card (e.g., a machine-to-machine SIM card, a Universal Integrated Circuit Card, without limitation).

The power source 110 is configured to supply power to the delivery system 112 and the various electronic components, such as the one or more processors 102, memory 104, communication equipment 108, and the like. Power source 110 may be, as a non-limiting example, a power storage device (e.g., a battery, without limitation), a power inlet, a power regulator, or combination thereof.

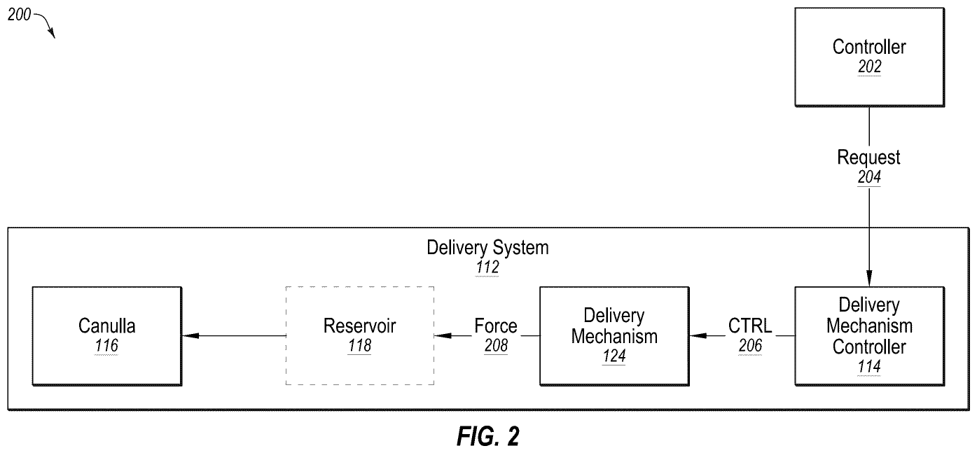

FIG. 2 is a block diagram of a medicament delivery system 200 for controlled administration of medicament to a user-body, in accordance with one or more examples.

The controller 202 is configured to manage automated medicament delivery device 100 and, more generally, administration of medicament to a user-body. In one or more embodiments, controller 202 may be implemented by instructions 106 and one or more processors 102 of the automated medicament delivery device 100 of FIG. 1.

In various embodiments, controller 202 and delivery system 112 may be realized in different devices (e.g., controller 202 may be realized in a physically different device (or devices)) than delivery system 112 is realized, or in the same device. When realized in different devices, functionality of controller 202 and delivery system 112 may be implemented, at least in part, by respective memory and one or more processors of their respective devices. When realized in a same device, functionality of controller 202 and delivery system 112 may be implemented, at least in part, by like memory and like one or more processors, respective memory and respective one or more processors, or a combination thereof. Non-limiting examples of devices in which controller 202, or a portion thereof, may be realized include: a handheld electronic computing device, such as a dedicated electronic device, a smart phone, a tablet computer, a wearable device (e.g., a smart watch, without limitation), a cloud computing device, and the like.

In various embodiments, the controller 202 may be configured to receive analyte data (e.g., from the analyte sensor, without limitation) including analyte values. In one or more embodiments, controller 202 may determine information about analytes within a user-body at least partially based on analyte data, for example, amounts, trends, distributions, without limitation. The controller 202 may analyze information about analytes in a user-body and may present the information and/or analysis to a patient, caregiver, or healthcare provider, as a non-limiting example, via an application (e.g., executing on a personal computer, smart phone, cloud server, or combinations thereof).

In various embodiments, the controller 202 may be configured to receive information from inputs from the patient or a caregiver (e.g., when the patient ate a meal or when the patient exercised, without limitation), and inputs from other electronic devices (e.g., information from a smart watch, without limitation) and to utilize such information as discussed herein. For example, in various embodiments, controller 202 may utilize some or a totality of such information to determine amounts of medicament to administer and timing of administration of medicament. Further, controller 202 may also be configured to determine requests, including request to administer dose 204, and send those requests to the automated medicament delivery device 100.

In various embodiments, controller 202 may be configured to determine a target dose amount to administer to a user of medicament delivery system 200. Controller 202 may determine a target dose amount at least partially based on therapy parameters, meal information, analyte values, and a control algorithm, without limitation.

In the context of insulin therapy to treat diabetes, therapy parameters may include insulin sensitivity factor (ISF), carbohydrate ratio (CR), amount of daily dose of long-acting insulin (LAI), doses of fast-acting or rapid-acting insulin, a current glucose value, and derivatives thereof without limitation. The timing and target dose amounts associated with requests generated by controller 202 may be governed by one or more control algorithms, discussed below.

Controller 202 may send a request to administer dose to delivery system 112, and more specifically, delivery mechanism controller 114.

The cannula 116 is insertable into a user-body (e.g., with a tip thereof positioned subcutaneously, without limitation) and is configured to provide medicament to a user-body (e.g., subcutaneously into the user-body, without limitation).

The reservoir 118 is configured to store and retain a medicament therein. As a non-limiting example, the reservoir 118 may be a hollow body, a flexible pouch, a chamber, a vial, without limitation. In various embodiments, reservoir 118 is a fluid reservoir for holding medicament and may be, as a non-limiting example, formed from the walls of a cartridge. In the cartridge example, delivery system 112 may include a chamber (i.e., a space or region defined within delivery system 112) configured to receive and hold a prefilled (prefilled with medicament) cartridge, eject an exhausted cartridge, and optionally receive a prefilled cartridge to replace (i.e., a replacement cartridge) the exhausted cartridge. Generally speaking, a volume of fluid in reservoir 118 will be greater in a pre-filled state than the volume in an exhausted state. Additionally or alternatively to the cartridge example, delivery system 112 is a multi-part delivery device where one of the two parts includes the reservoir 118 and the other one of the two parts includes the delivery mechanism controller 114. The other one of the two parts may optionally further include controller 202. Either one of the two parts may optionally include delivery mechanism 124 (e.g., a piston pump, without limitation). The one of the two parts that includes reservoir 118 is disposable (i.e., a “disposable part”) and configured to be removable secured to the other part of medicament delivery system 200. When reservoir 118 is exhausted, the disposable part may be removed and a replacement part including a reservoir 118 optionally in a pre-filled state.

Delivery mechanism 124 is configured to urge fluid in reservoir 118 toward an interface for dispensing fluid (interface not shown). In various embodiments, delivery mechanism 124 may be positioned adjacent to reservoir 118. The delivery mechanism 124 is configured to cause an amount of the medicament to be administered to the user-body by causing the amount to flow from the reservoir 118 toward and into a user-body via cannula 116, which is in fluidic communication with the reservoir 118. In various embodiments, delivery mechanism 124 may utilize any suitable mechanism to generate positive displacement or negative displacement to transfer amounts of medicament from reservoir 118 toward cannula 116 and a user-body.

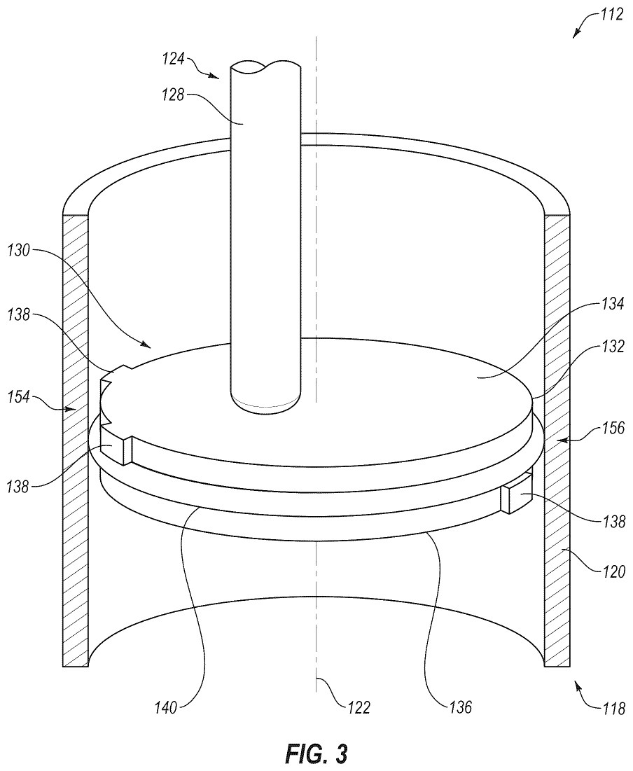

For example, delivery mechanism 124 may apply a force to a piston 130 (or similar mechanisms, refer to FIGS. 3-8) free to move within reservoir 118, and via such a force, move the piston 130 in a direction that urges fluid in reservoir 118 toward the aforementioned interface. In one or more examples, delivery mechanism 124 may include an electrical motor (e.g., an AC or DC motor) that produces a force to, directly or indirectly, move the piston 130 to perform a delivery action. A delivery action may dispense at a predetermined rate (i.e., a predictable amount of fluid over a predictable duration of time). The delivery mechanism 124 may be capable of multiple rates of delivery, and in one or more examples, may be preconfigured to use a same rate of delivery all the time, or may delivery discrete doses of medicament as controlled by controller 202 and/or as directed by a user to deliver a particular dose amount.

Such an electric motor may be a current controlled electric motor, voltage controlled electric motor, pulse-width controlled electric motor, or combination or sub combination thereof. Such an electronic motor may be directly or indirectly digitally controlled. The control signal 206 may be determined and generated by delivery mechanism controller 114 to correspond to a delivery action. A control signal 206 may also be referred to herein as a “command 206” or an “instruction 206.”

Delivery mechanism controller 114 may generate control signals 206 corresponding to one or more delivery actions at least partially based on a request to administer dose 204 received from controller 202. Control signal 206 may include first control signals to cause delivery mechanism 124 to generate resultant force 208, and a second, different control signal to cause delivery mechanism 124 to not or stop generating force 208. Utilizing control signals 206, delivery mechanism controller 114 may control a length of a duration of time that delivery mechanism 124 produces force 208 and applies it to dispense fluid from reservoir 118, and indirectly, an amount of fluid dispensed from reservoir 118.

When delivery mechanism controller 114 generates control signal 206 in response to a request to administer dose 204 from controller 202, it may generate the control signal 206 at least partially based on a value of a target dose amount included with, or indicated by, request to administer dose 204. One or more delivery actions may be utilized to dispense an amount of fluid corresponding to a dose amount determined by controller 202. For example, a fluid amount dispensed according to a delivery action may be less than a dose amount. Generally speaking, the delivery mechanism 124 and delivery system 112 are agnostic to the purpose for which fluid is dispensed and unaware of what constitutes a working amount of fluid to administer a dose, or series of doses, of medicament. So, while it may be desirable that a fluid amount dispensed according to one or more delivery actions will be exactly the same as a target dose amount, some negligible difference is specifically contemplated, and what is considered “negligible” will depend on specific operation conditions.

In one or more examples, delivery mechanism controller 114 may be configured to determine and generate feedback information about delivery actions, such as times of delivery actions and dispensed amounts, without limitation. Feedback information may be generated based on information generated by delivery mechanism 124 or by sensors utilized by delivery mechanism controller 114 to monitor operation of delivery mechanism 124 (sensors not depicted). For example, sensors to monitor mechanical movement, current consumption, a voltage profile of an electric motor, without limitation. Such information may be logged and provided to and stored at controller 202, without limitation, for e.g., later processing or reading, without limitation. For example, the logs can be processed to determine patterns that may be utilized to determine whether delivery system 112 is operating as expected (e.g., in a predictable manner, without limitation), and if a difference between actual and expected operation exceeds a threshold, delivery mechanism controller 114 may be updated (e.g., firmware, parameters, or both, of delivery mechanism controller 114 may be updated, without limitation) to compensate or correct for the difference. Additionally or alternatively to updating the firmware or parameters, in a multi-part system, one or more parts including delivery mechanism controller 114 may be indicated as needing replacement (e.g., an alarm or alert is generated at delivery system 112, medicament delivery system 200, a mobile device or computer in communication therewith, without limitation).

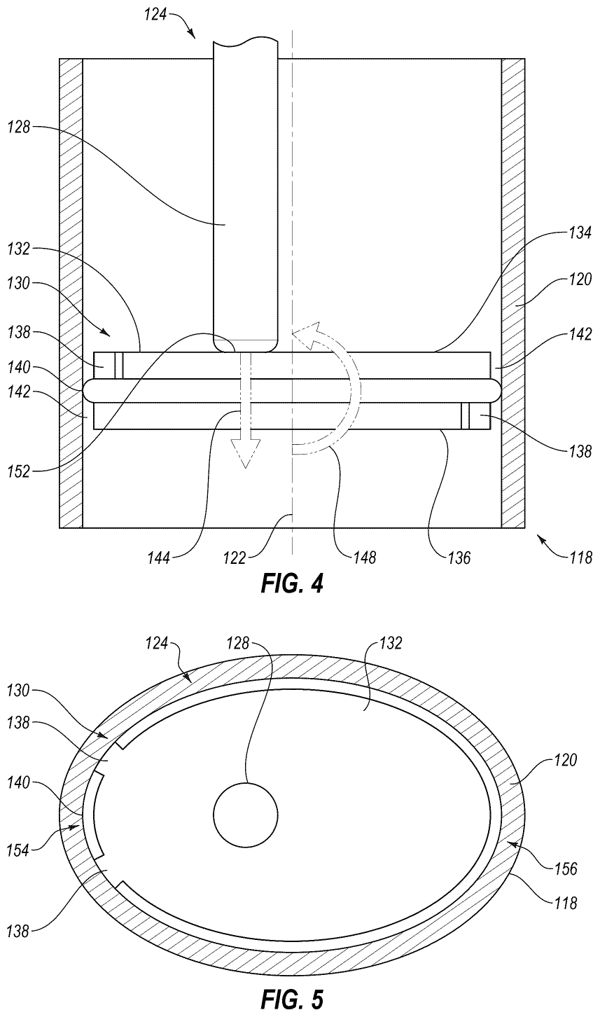

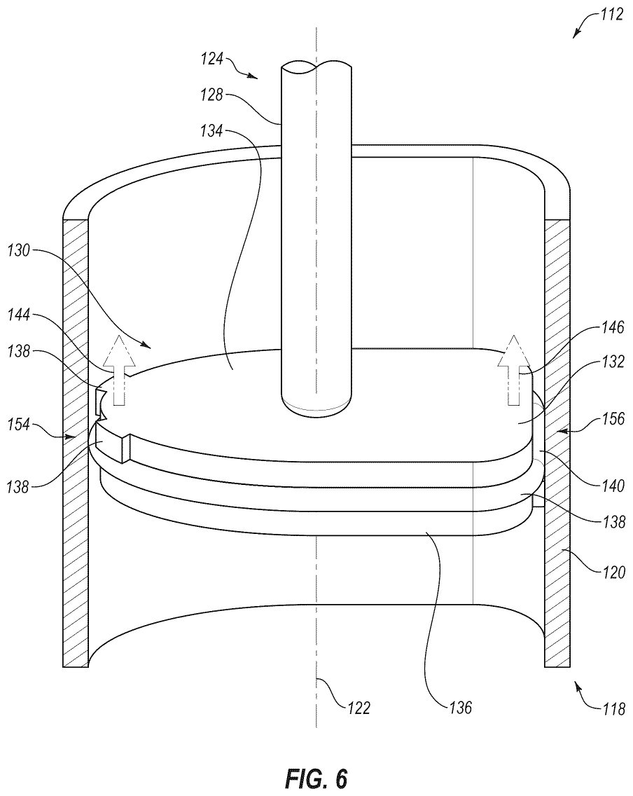

FIG. 3 is a cut-away perspective view of a portion of a delivery system 112 in accordance with one or more embodiments. FIG. 4 is a cut-away side view of the portion of the delivery system 112 of FIG. 3. FIG. 5 is a top view of the portion of the delivery system 112 of FIG. 3. The reservoir 118 includes a sidewall 120 including a hollow structure defining an internal volume therein. The hollow structure may be a hollow cylinder (e.g., a right hollow elliptical cylinder, a right hollow circular cylinder, without limitation), a hollow prism (e.g., a right hollow prism, such as a cuboid, or a hollow prism with rounded corners, without limitation), a stadium shape, or other similar shapes. The internal volume includes a first cross-sectional shape. In the embodiment illustrated in FIGS. 3-5, the first cross-sectional shape is a symmetrical cross-section (e.g., a right hollow elliptical cylinder, without limitation). In other embodiments, the first cross-sectional shape is an asymmetrical shape (as will be described in further detail below with reference to FIGS. 6-8).

The delivery mechanism 124 includes a piston 130, a seal 140, and a rod 128. The piston 130 is received within the internal volume of the reservoir 118. The piston 130 includes a body 132 and at least one protrusion 138 (e.g., tab, projection, bump, or overhang, without limitation) extending from the body 132. The body 132 includes a second cross-sectional shape substantially similar to the first cross-sectional shape. The second cross-sectional shape is smaller than the first cross-sectional shape (e.g., an area and/or footprint, without limitation).

The body 132 includes a connection end 134 connected to the rod 128 and a pressure end 136 defining a containment volume with the reservoir 118 for containing medicament therein. The body 132 is configured to receive the seal 140 around a perimeter thereof between the connection end 134 and the pressure end 136. The seal 140 extends outward from the rod beyond the perimeter of the body 132 and forms a seal (i.e., a continuous and reliably effective barrier) between the body 132 and the sidewall 120 of the reservoir 118. This configuration ensures that the seal 140 maintains direct contact with both the outer surface of the body 132 and the inner surface of the sidewall 120, effectively preventing liquid bypass and ensuring controlled delivery. Notably, there may be some inaccuracies as the plunger stabilizes into its biased profile during a priming cycle, for example when the pump is expelling air from the system and the cannula is not in a user-body. The body 132 may be positioned within the reservoir 118 with one or more gaps 142 defined between the perimeter of the body and the inner surface of the sidewall 120, while the seal 140 contacts both the body 132 and the sidewall 120 to form the seal therebetween. The one or more gaps 142 are deliberately maintained between the perimeter of the body 132 and the inner sidewall 120 of the reservoir 118 to create, at least in part, an imbalanced friction during movement of the piston 130, as described below.

The reservoir 118 and delivery mechanism 124 are configured to cause an imbalanced friction (e.g., a controlled friction bias, without limitation) between the seal 140 and the sidewall 120, and more specifically around the perimeter of the body 132, during movement of the piston 130 relative to the reservoir 118. The imbalanced friction applies a moment 148 to the piston 130 as the piston 130 progresses linearly within the reservoir 118. The moment 148 (e.g., torque, without limitation) may prevent (e.g., reduce or counteract at least in part, without limitation) walking/wobbling of the piston 130 (e.g., lateral or rotational motion, without limitation) as the piston 130 moves within the reservoir 118, and ensure the piston 130 reliably remains aligned and stable during operational movement.

The friction bias may be added in several ways. In various embodiments, at least a portion of the friction bias is caused by a contact point 152 of the rod 128 (or a center point of where the rod pushes on piston 130) being offset from a center (e.g., center of mass) of the piston 130. By offsetting the drive force contact point 152 from a center of the piston 130, the rod 128 will apply a known moment 148 (e.g., magnitude and direction of the moment 148 are known or predetermined, without limitation) to the piston 130 creating the friction bias. The friction bias may be caused by a perimeter of a second side 156 of the body 132 being longer than a first side 154 of the body 132 where the division between the second side 156 and the first side 154 is relative to the contact point 152.

The at least one protrusion 138 is configured and positioned to maintain axial alignment of the piston 130 within the cylindrical (e.g., an elliptical cylindrical shape, without limitation) reservoir 118 and counterbalance the applied known moment 148. Each of the at least one protrusion 138 extends from the body 132 towards the sidewall 120 and counteracts the known moment 148 applied to the piston 130 and prevents rotation of the piston 130. In various embodiments, the at least one protrusion 138 extends from the body and contacts the sidewall 120. Each of the at least one protrusion 138 is configured to slide along the sidewall 120 while preventing rotation of the piston 130 while the piston 130 moves in a first direction. Movement in the first direction reduces a medicament volume defined by the reservoir 118 and the piston 130. In various embodiments, the piston includes multiple protrusions 138. In various embodiments, the protrusion 138 or protrusions 138 are only positioned in locations necessary to counteract the moment 148 (since the direction of the moment 148 is known) while the piston moves in the first direction.

In various embodiments, one or more protrusions 138 extends from the body 132 adjacent to the connection end 134 of the body 132 (e.g., sharing a border with the connection end 134, about sharing a border with the connection end 134, or closer to the connection end 134 than the pressure end 136, without limitation) and at a first side 154 of the body 132, and/or one or more protrusions 138 extends from the body 132 adjacent to the pressure end 136 of the body 132 (e.g., sharing a border with the pressure end 136, about sharing a border with the pressure end 136, or closer to the pressure end 136 than the connection end 134, without limitation) and at a second side 156 of the body 132, the first side 154 being opposite the second side 156 and closer to the contact point 152 than the second side 156. The second side 156 is a region of the body 132 furthest/distal to the contact point 152 and the first side 154 is a region of the body 132 closest/proximal to the contact point 152.

The one or more protrusions 138 on the second side 156 may be in substantially similar positions as the one or more protrusions 138 on the first side 154 on opposite ends of the body 132. The one or more protrusions 138 are each offset from a vertical center of the body 132 so that the force applied to the one or more protrusions 138 by the sidewall 120 counteract the moment 148. In various embodiments, the one or more protrusions 138 on the second side 156 includes multiple protrusions 138 that are symmetrically positioned relative to an axis of symmetry of the body 132, and the one or more protrusions 138 on the first side 154 includes multiple protrusions 138 that are symmetrically positioned relative to an axis of symmetry of the body 132. Symmetrically positioning multiple protrusions 138 on each side of the body 132 may further stabilize the piston 130 by countering any moments that occur about an axis transverse to an axis of the moment 148.

A cumulative effect of the above is to provide a more consistent and controlled delivery of medicament, reducing risk of dosage variance due to piston misalignment or erratic movement.

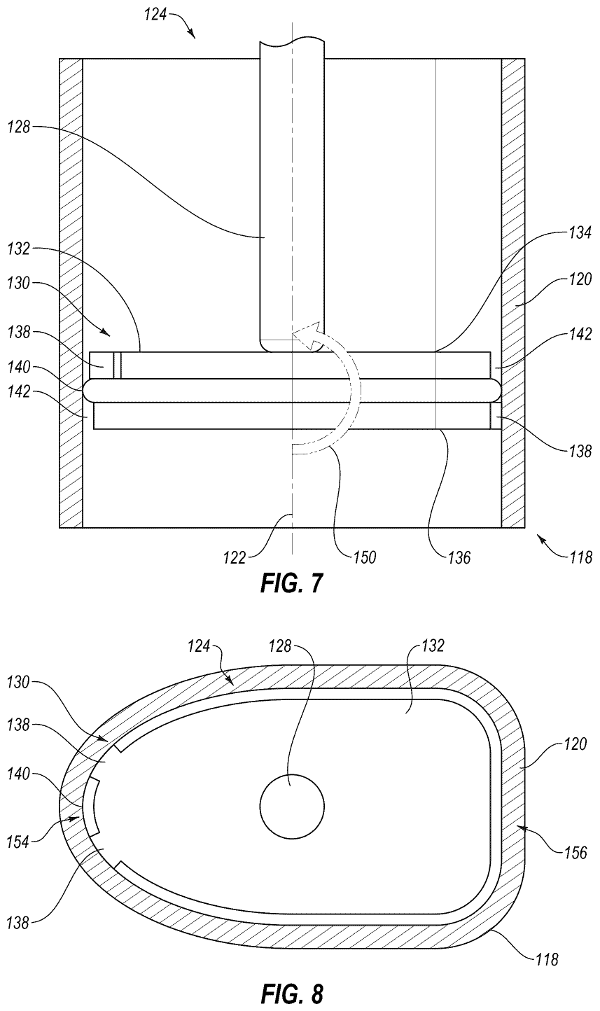

FIG. 6 is a cut-away perspective view of a portion of a delivery system 112 in accordance with one or more embodiments. FIG. 7 is a cut-away side view of the portion of the delivery system 112 of FIG. 6. FIG. 8 a top view of the portion of the delivery system 112 of FIG. 6. Referring to FIGS. 6-8, in various embodiments, at least a portion of the friction bias is caused by imbalanced friction between sides of the piston 130. In these embodiments, the piston 130 includes an asymmetrical shape. With the asymmetrical shape, a perimeter of a second side 156 of the body 132 includes a length that is longer than a length of a perimeter of a first side 154 of the body 132 with the division between the second side 156 and the first side 154 is relative to the center 122 of the body 132. With the different length perimeters, a first force 144 caused by friction between the seal 140 and the sidewall 120 along the first side 154 is smaller than a second force 146 caused by friction between the seal 140 and the wall along the second side 156. The first force 144 being less than the second force 146 imparts a moment on the piston 130.

As discussed above, the piston 130 includes protrusions 138 extending from the body 132 and positioned to contact the sidewall 120 of the reservoir 118 and counteract the moment 150 applied to the piston 130 and to prevent rotation of the piston 130.

In various embodiments, one or more protrusions 138 extends from the body 132 at the pressure end 136 of the body 132 and at a side with a higher friction force (e.g., the second side 156 of the body 132), and one or more protrusions 138 extends from the body 132 at the connection end 134 of the body 132 and at a side with a lower friction force (e.g., the first side 154 of the body 132).

Due to the asymmetry of the body 132, the one or more protrusions 138 on the second side 156 may be in different positions as the one or more protrusions 138 on the first side 154. The protrusions 138 are each offset from a vertical center of the body 132 so that the force applied to the protrusions 138 by the sidewall 120 counteract the moment 150.

In various embodiments, the friction bias is caused by both a contact point 152 of the rod 128 being offset from a center (e.g., geometric center and center of mass) of the piston 130 and the body 132 including an asymmetrical shape where a perimeter of a second side 156 of the body 132 includes a length that is longer than a length of a perimeter of a first side 154 of the body 132 (e.g., the perimeter of one of the first side 154 or the second side 156 exhibits an elongated length compared to the other, without limitation). The division between the second side 156 and the first side 154 is relative to the center 122 of the body 132. In these various embodiments, the one or more protrusions 138 extending from the body 132 at the pressure end 136 of the body 132 and the one or more protrusions 138 extending from the body 132 at the connection end 134 of the body 132 are positioned to counteract the combination of the moment 148 and the moment 150.

While the asymmetrical shape is described relative to the first side 154 and the second side 156 of the piston 130 illustrated in FIGS. 6-8, in various embodiments, the asymmetrical shape is oriented transverse to a direction of the offset of the contact point 152 from the center of the piston 130. In these various embodiments, the first side 154 of the piston illustrated in FIGS. 6-8 may be considered a third side and the second side 156 may be considered a fourth side.

Non-limiting example embodiments of the present disclosure may include:

Embodiment 1: A delivery system of an automated medicament delivery device for automated administration of medicament to a user-body, the delivery system comprising: a reservoir including a sidewall defining an internal volume therein; and a delivery mechanism comprising: a piston received in the internal volume, the piston including: a body, a seal received around a perimeter of the body and extending outward beyond the perimeter and forming a seal between the body and the sidewall, and at least one protrusion, each of the at least one protrusion extending from the body beyond the perimeter and towards the sidewall; and a rod connected to the body of the piston, the delivery mechanism configured to cause a friction bias between the seal and the sidewall around the perimeter of the body of the piston during movement of the piston relative to the reservoir.

Embodiment 2: The delivery system according to Embodiment 1, wherein the friction bias applies a moment to the piston during movement of the piston and the at least one protrusion is positioned to counteract the moment and prevent rotation of the piston while the piston moves in a first direction, movement in the first direction reducing a medicament volume defined by the reservoir and the piston.

Embodiment 3: The delivery system according to Embodiments 1 and 2, wherein each of the at least one protrusion extends from the body of the piston and contacts the sidewall.

Embodiment 4: The delivery system according to any of Embodiments 1 to 3, wherein: the body of the piston includes a connection end and a pressure end, opposite the connection end, the rod connected to the body of the piston at a connection point on the connection end, and the connection point being offset from a center of the body of the piston to a first side of the body of the piston; and the at least one protrusion includes multiple protrusions with one or more first protrusions extending from the first side of the body of the piston adjacent to the connection end and a one or more second protrusions extending from a second side of the body of the piston, opposite the first side, adjacent to the pressure end.

Embodiment 5: The delivery system according to any of Embodiments 1 to 4, wherein the body of the piston includes an asymmetrical shape, and wherein the connection point being offset from a center of the body of the piston is configured to cause at least a first portion of the friction bias and the asymmetrical shape of the body of the piston is configured to cause at least a second portion of the friction bias.

Embodiment 6: The delivery system according to any of Embodiments 1 to 5, wherein a perimeter of a third side of the body of the piston includes a length that is longer than a length of a perimeter of a fourth side of the body of the piston.

Embodiment 7: The delivery system according to any of Embodiments 1 to 6, wherein the body of the piston includes an asymmetrical shape with a perimeter of a first side of the body of the piston including a length that is longer than a length of a perimeter of a second side of the body of the piston, a division between the first side and the second side is relative to a center of the body of the piston.

Embodiment 8: The delivery system according to any of Embodiments 1 to 7, wherein a first force caused by friction between the seal and the sidewall along the first side is smaller than a second force caused by friction between the seal and the sidewall along the second side resulting in a known moment applied to the piston during movement thereof.

Embodiment 9: The delivery system according to any of Embodiments 1 to 8, wherein the at least one protrusion is configured to slide along the sidewall while preventing rotation of the piston while the piston moves in a first direction, movement in the first direction reducing a medicament volume defined by the reservoir and the piston.

Embodiment 10: A delivery system of an automated medicament delivery device for automated administration of medicament to a user-body, the delivery system comprising: a reservoir including a sidewall defining an internal volume therein; and a delivery mechanism comprising: a piston received in the internal volume, the piston including: a body including a connection end and a pressure end, opposite the connection end, a seal received around a perimeter of the body of the piston and extending outward beyond the perimeter and forming a seal between the body of the piston and the sidewall, one or more first protrusions extending from a first side of the body of the piston adjacent to the connection end and extending toward the sidewall, and one or more second protrusions extending from a second side of the body of the piston, opposite the first side, adjacent to the pressure end and extending toward the sidewall; and a rod connected to the body of the piston at a connection point on the connection end, the connection point being offset from a center of the body of the piston to the first side of the body of the piston.

Embodiment 11: The delivery system according to Embodiment 10, wherein each of the plurality of protrusions extends from the body of the piston and contacts the sidewall.

Embodiment 12: The delivery system according to Embodiments 10 and 11, wherein the plurality of protrusions are configured to slide along the sidewall while preventing rotation of the piston while the piston moves in a first direction, movement in the first direction reducing a medicament volume defined by the reservoir and the piston.

Embodiment 13: The delivery system according to any of Embodiments 10 to 12, wherein the internal volume includes a first cross-sectional shape and the body of the piston includes a second cross-sectional shape substantially similar to the first cross-sectional area, the second cross-sectional area is smaller than the first cross-sectional shape.

Embodiment 14: The delivery system according to any of Embodiments 10 to 13, wherein the one or more second protrusions on the second side include multiples protrusions that are symmetrically positioned relative to an axis of symmetry of the body of the piston and the one or more first protrusions on the first side includes multiple protrusions that are symmetrically positioned relative to the axis of symmetry of the body of the piston.

Embodiment 15: The delivery system according to any of Embodiments 10 to 14, wherein the body of the piston includes an asymmetrical shape, and wherein the connection point being offset from a center of the body of the piston is configured to cause at least a first portion of the friction bias and the asymmetrical shape of the body of the piston is configured to cause at least a second portion of the friction bias.

Embodiment 16: The delivery system according to any of Embodiments 10 to 15, wherein a perimeter of a third side of the body of the piston includes a length that is longer than a length of a perimeter of a fourth side of the body of the piston.

Embodiment 17: A delivery system of an automated medicament delivery device for automated administration of medicament to a user-body, the delivery system comprising: a reservoir including a sidewall defining an internal volume therein; and a delivery mechanism comprising a piston received in the internal volume, the piston comprising: a body including a connection end and a pressure end, opposite the connection end, the body of the piston including an asymmetrical shape with a first side and a second side, a first perimeter of the first side of the body of the piston including a length that is longer than a length of a second perimeter of the second side of the body of the piston; a seal received around a combined perimeter of the body of the piston and extending outward beyond the combined perimeter and forming a seal between the body of the piston and the sidewall; one or more first protrusions extending from the first side of the body of the piston adjacent to the connection end and extending toward the sidewall; and one or more second protrusions extending from a second side of the body of the piston, opposite the first side, adjacent to the pressure end and extending toward the sidewall.

Embodiment 18: The delivery system according to Embodiment 17, wherein each of the plurality of protrusions extends from the body of the piston and contacts the sidewall.

Embodiment 19: The delivery system according to Embodiments 17 and 18, wherein the plurality of protrusions are configured to slide along the sidewall while preventing rotation of the piston while the piston moves in a first direction, movement in the first direction reducing a medicament volume defined by the reservoir and the piston.

Embodiment 20: The delivery system according to Embodiments 17 to 19, wherein a first force caused by friction between the seal and the sidewall along the first side is smaller than a second force caused by friction between the seal and the sidewall along the second side resulting in a known moment applied to the piston during movement thereof.

The embodiments of the disclosure described above and illustrated in the accompanying drawings do not limit the scope of the disclosure, which is encompassed by the scope of the appended claims and their legal equivalents. Any equivalent embodiments are within the scope of this disclosure. Indeed, various modifications of the disclosure, in addition to those shown and described herein, such as alternate useful combinations of the elements described, will become apparent to those skilled in the art from the description. Such modifications and embodiments also fall within the scope of the appended claims and equivalents.

What is claimed is:

1. A delivery system of an automated medicament delivery device for automated administration of medicament to a user-body, the delivery system comprising:
a reservoir including a sidewall defining an internal volume therein; and
a delivery mechanism comprising:
a piston received in the internal volume, the piston including:
a body,
a seal received around a perimeter of the body and extending outward beyond the perimeter and forming a seal between the body and the sidewall, and
at least one protrusion extending from the body beyond the perimeter and towards the sidewall; and
a rod connected to the body of the piston, the delivery mechanism configured to cause a friction bias between the seal and the sidewall around the perimeter of the body of the piston during movement of the piston relative to the reservoir.

2. The delivery system of claim 1, wherein the friction bias applies a moment to the piston during movement of the piston and the at least one protrusion is positioned to counteract the moment and prevent rotation of the piston while the piston moves in a first direction, movement in the first direction reducing a medicament volume defined by the reservoir and the piston.

3. The delivery system of claim 1, wherein the at least one protrusion extends from the body of the piston and contacts the sidewall.

4. The delivery system of claim 1, wherein:
the body of the piston includes a connection end and a pressure end, opposite the connection end, the rod connected to the body of the piston at a connection point on the connection end, and the connection point being offset from a center of the body of the piston to a first side of the body of the piston; and
the at least one protrusion includes multiple protrusions with one or more first protrusions extending from the first side of the body of the piston adjacent to the connection end and a one or more second protrusions extending from a second side of the body of the piston, opposite the first side, adjacent to the pressure end.

5. The delivery system of claim 4, wherein the body of the piston includes an asymmetrical shape, and wherein the connection point being offset from a center of the body of the piston is configured to cause at least a first portion of the friction bias and the asymmetrical shape of the body of the piston is configured to cause at least a second portion of the friction bias.

6. The delivery system of claim 5, wherein a perimeter of a third side of the body of the piston includes a length that is longer than a length of a perimeter of a fourth side of the body of the piston.

7. The delivery system of claim 1, wherein the body of the piston includes an asymmetrical shape with a perimeter of a first side of the body of the piston including a length that is longer than a length of a perimeter of a second side of the body of the piston, a division between the first side and the second side is relative to a center of the body of the piston.

8. The delivery system of claim 1, wherein a first force caused by friction between the seal and the sidewall along a first side is smaller than a second force caused by friction between the seal and the sidewall along a second side resulting in a known moment applied to the piston during movement thereof.

9. The delivery system of claim 1, wherein the at least one protrusion is configured to slide along the sidewall while preventing rotation of the piston while the piston moves in a first direction, movement in the first direction reducing a medicament volume defined by the reservoir and the piston.

10. A delivery system of an automated medicament delivery device for automated administration of medicament to a user-body, the delivery system comprising:
a reservoir including a sidewall defining an internal volume therein; and
a delivery mechanism comprising:
a piston received in the internal volume, the piston including:
a body including a connection end and a pressure end, opposite the connection end,
a seal received around a perimeter of the body of the piston and extending outward beyond the perimeter and forming a seal between the body of the piston and the sidewall,
one or more first protrusions extending from a first side of the body of the piston adjacent to the connection end and extending toward the sidewall, and
one or more second protrusions extending from a second side of the body of the piston, opposite the first side, adjacent to the pressure end and extending toward the sidewall; and
a rod connected to the body of the piston at a connection point on the connection end, the connection point being offset from a center of the body of the piston to the first side of the body of the piston.

11. The delivery system of claim 10, wherein each of the plurality of protrusions extends from the body of the piston and contacts the sidewall.

12. The delivery system of claim 10, wherein the plurality of protrusions are configured to slide along the sidewall while preventing rotation of the piston while the piston moves in a first direction, movement in the first direction reducing a medicament volume defined by the reservoir and the piston.

13. The delivery system of claim 10, wherein the internal volume includes a first cross-sectional shape and the body of the piston includes a second cross-sectional shape substantially similar to the first cross-sectional shape, the second cross-sectional shape is smaller than the first cross-sectional shape.

14. The delivery system of claim 10, wherein the one or more second protrusions on the second side are symmetrically positioned relative to an axis of symmetry of the body of the piston and the one or more first protrusions on the first side are symmetrically positioned relative to the axis of symmetry of the body of the piston.

15. The delivery system of claim 10, wherein the body of the piston includes an asymmetrical shape, and wherein the connection point being offset from a center of the body of the piston is configured to cause at least a first portion of a friction bias and the asymmetrical shape of the body of the piston is configured to cause at least a second portion of the friction bias.

16. The delivery system of claim 15, wherein a perimeter of a third side of the body of the piston includes a length that is longer than a length of a perimeter of a fourth side of the body of the piston.

17. A delivery system of an automated medicament delivery device for automated administration of medicament to a user-body, the delivery system comprising:
a reservoir including a sidewall defining an internal volume therein; and
a delivery mechanism comprising a piston received in the internal volume, the piston comprising:
a body including a connection end and a pressure end, opposite the connection end, the body of the piston including an asymmetrical shape with a first side and a second side, a first perimeter of the first side of the body of the piston including a length that is longer than a length of a second perimeter of the second side of the body of the piston;
a seal received around a combined perimeter of the body of the piston and extending outward beyond the combined perimeter and forming a seal between the body of the piston and the sidewall;
one or more first protrusions extending from the first side of the body of the piston adjacent to the connection end and extending toward the sidewall; and
one or more second protrusions extending from a second side of the body of the piston, opposite the first side, adjacent to the pressure end and extending toward the sidewall.

18. The delivery system of claim 17, wherein each of the plurality of protrusions extends from the body of the piston and contacts the sidewall.

19. The delivery system of claim 17, wherein the plurality of protrusions are configured to slide along the sidewall while preventing rotation of the piston while the piston moves in a first direction, movement in the first direction reducing a medicament volume defined by the reservoir and the piston.

20. The delivery system of claim 17, wherein a first force caused by friction between the seal and the sidewall along the first side is smaller than a second force caused by friction between the seal and the sidewall along the second side resulting in a known moment applied to the piston during movement thereof.
